(19) United States
(12) Patent Application Publication
(10) Pub. No.: US 2007/0219507 A1
(43) Pub. Date: Sep. 20, 2007

(54) PHARMACEUTICAL CARTRIDGE PISTON WITH RIGID CORE

(76) Inventors: He Dai, Carmel, IN (US); Douglas James Allen, Indianapolis, IN (US)

Correspondence Address: ELI LILLY & COMPANY, PATENT DIVISION, P.O. BOX 6288, INDIANAPOLIS, IN, 46206-6288, US

(21) Appl. No.: 10/599,639

(22) PCT Filed: Mar. 29, 2005
(86) PCT No.: PCT/US05/10148

§ 371 Date: Oct. 4, 2006

Related U.S. Application Data
(60) Provisional application No. 60/560,559, filed on Apr. 8, 2004.

(51) Int. Cl. A61M 5/315

(52) U.S. Cl. 604/218

(57) ABSTRACT

A pharmaceutical cartridge piston including a rigid core to resist compression of the piston. The resistance to compression contributes to the piston providing dose accuracy and dynamic recovery advantages.

### BACKGROUND OF THE INVENTION

The present invention pertains to medication delivery devices, and, in particular, to a piston of a pharmaceutical cartridge for a medication delivery device.

A variety of medication delivery devices, such as injector pens and infusion pumps, employ pharmaceutical cartridges that include a movable piston and contain a multi-dose quantity of liquid medication. In an injector pen, a drive member, extending from within a base of the injector pen and operably connected with typically more rearward mechanisms of the pen that control drive member motion, is movable forward to advance the piston in the cartridge in such a manner to dispense the contained medication from an outlet at the opposite cartridge end, typically through a needle that penetrates a stopper or septum at that opposite end. In disposable pens, after a pen has been utilized to exhaust the supply of medication within the cartridge, the entire pen is discarded by a user, who then begins using a new replacement pen. In reusable pens, after a pen has been utilized to exhaust the supply of medication within the cartridge, the pen is disassembled to allow replacement of the spent cartridge with a fresh cartridge, and then the pen is reassembled for its subsequent use.

While these types of medication delivery devices with cartridges offer a number of advantages to their users, such devices are not without their limitations. For example, possible compression of the cartridge piston during an injection can have a negative effect on the overall operation of the device. For an injector pen, such piston compression can result in the pen's medication drooling from the needle after an injecting force has been removed and the needle has been prematurely withdrawn from the user, and/or the pen delivering a dose that is less than anticipated.

Thus, it would be desirable to provide an apparatus that can overcome one or more of these and other shortcomings of the prior art.

### BRIEF SUMMARY OF THE INVENTION

In one form thereof, the present invention provides a piston for a pharmaceutical cartridge including a tubular barrel extending in an axial direction, the piston being advanceable by a separate drive member of a medication delivery device equipped with the cartridge. The piston includes a body and a core. The body has a distal end, a proximal end and a sealing periphery, which distal end is in contact with a medication disposed within the cartridge barrel, which sealing periphery is in sealing contact with an interior surface of the barrel, and the distal end and the sealing periphery are unitarily constructed from a material having a first hardness. The core is within the body and sealed within the cartridge barrel between the distal end and the proximal end. The core is constructed from at least one material having a second hardness greater than the first hardness to limit axial compressibility of the piston.

One advantage of the present invention is that a pharmaceutical cartridge piston can be provided which resists axial compression during an injection.

Another advantage of the present invention is that a pharmaceutical cartridge piston can be provided which may enhance dose accuracy.

Still another advantage of the present invention is that a pharmaceutical cartridge piston can be provided which may reduce injection hold time.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taking in conjunction with the accompanying drawings, wherein:

FIG. 1 is a front perspective view of a pharmaceutical cartridge including a first embodiment of a piston of the present invention;

FIG. 2 is a longitudinal cross-sectional view of the cartridge of FIG. 1, which cartridge is further shown loaded in one form of an abstractly shown medication delivery device;

FIG. 3 is a rear perspective view of the piston of FIG. 1 removed from the rest of the cartridge;

FIG. 4 is a front perspective view in longitudinal cross-section of the piston of FIG. 3;

FIG. 5 is an exploded, front perspective view of the piston of FIG. 3; and

FIG. 6 is a longitudinal cross-sectional view of a second embodiment of a pistoin of the present invention.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE INVENTION

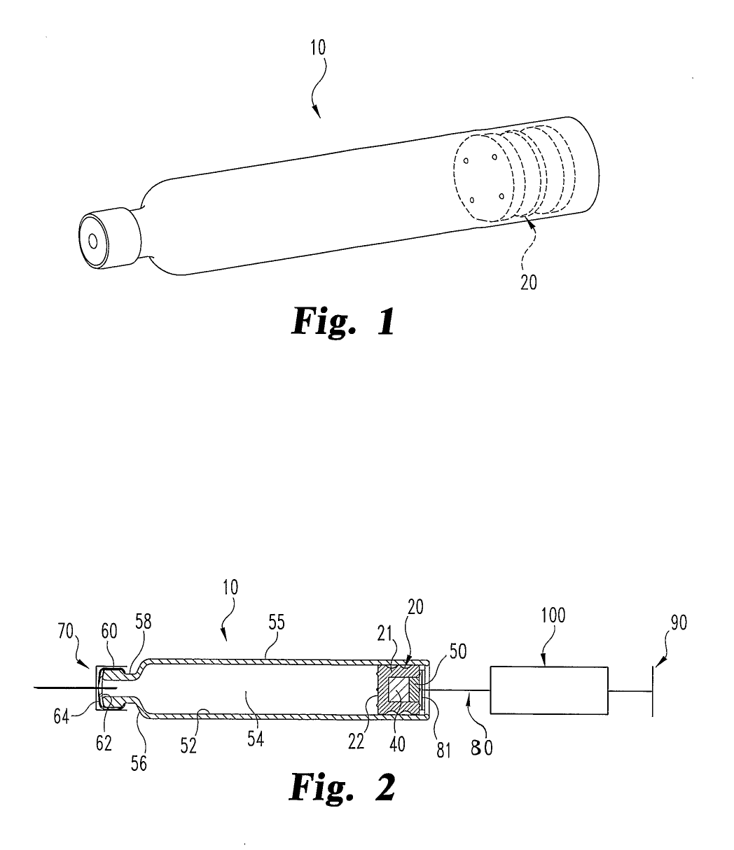

Referring now to FIG. 1, there is shown a perspective view of a pharmaceutical cartridge, generally designated 10, including a first embodiment of a piston of the present invention, which piston is generally designated 20. FIG. 2 shows the cartridge of FIG. 1 loaded in one type of a medication delivery device, such as an injector pen or injection pen. The present invention alternatively may be utilized in other medication delivery devices.

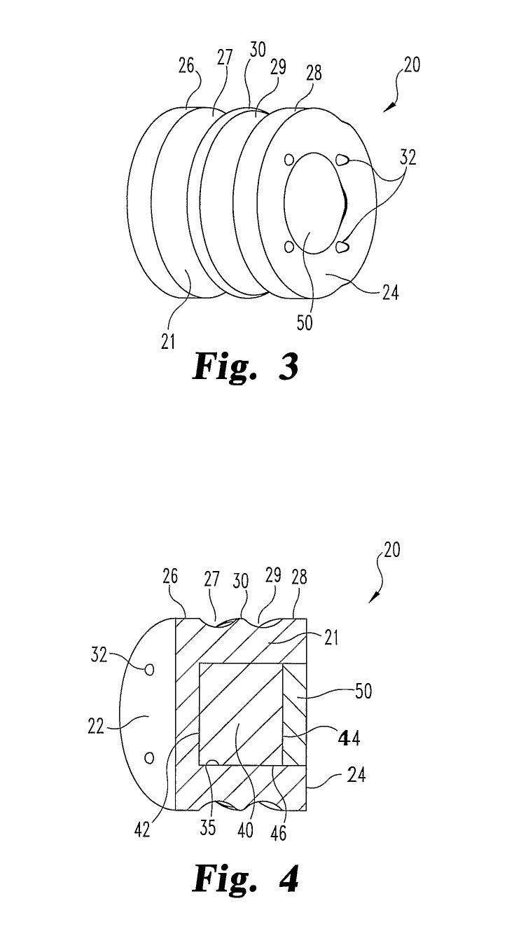

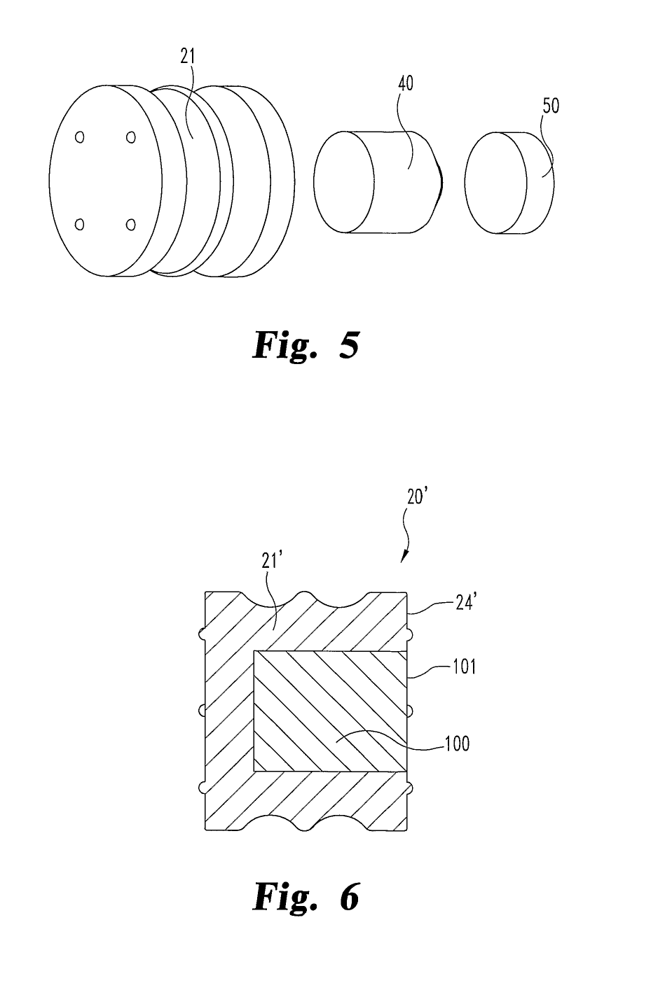

With additional reference to FIGS. 3-5, cartridge piston 20 has an axially extending, generally cylindrical, main body 21 that includes a distal end face 22 and a proximal end face 24. Along its radial periphery, body 21 is formed with a first sealing rib 26 that is immediately adjacent to the distal end face 22, a second sealing rib 28 immediately adjacent to the proximal end face 24, and an axially shorter rib 30 centrally disposed therebetween. Circumferential recesses 27 and 29 are disposed between ribs 26 and 30, and ribs 30 and 28, respectively. Sealing ribs 26, 28 and 30 each extend continuously around the circumference of body 21 to form rings that provide a fluid tight seal with the interior surface of the cartridge tubular barrel to prevent passage of materials out from and into the medicine reservoir. A plurality of protuberances 32 project from the planer distal end face 22 and the planer proximal end face 24 to limit adhesion between multiple pistons when in, for example, a hopper during assembly line production of multiple pharmaceutical cartridges.

Main body 21 is formed of a material, such as halo butyl rubber, that is chemically and biologically compatible with the not shown medication contained within cartridge 10. A silicone emulsion coating may be provided on body 21 to provide lubrication. One particularly suitable elastomeric formulation for body 21 is available from West Pharmaceutical Services of Lionville, Pa. and is known as West Formulation PH 4002/50 Red. This elastomeric formulation has a hardness, or resistance to deformation by indentation, using the Shore A Durometer scale, of between 45 and 55.

Piston 20 also includes a relatively rigid core or insert 40 that is fixedly disposed within a cylindrical hollow or bore 35 of piston body 21. Core 40 is shown as being cylindrical in overall form, with a planer distal face 42, a planer proximal face 44, and an outer periphery 46. Core 40 is axially centered within the axial or longitudinal length of body 21, and further is radially centered within body 21 so as to be in alignment with and symmetrical with respect to the central axis of cartridge tubular barrel 55.

Core 40 results in piston 20 being less compressible in the axial direction, while not compromising the ability of the piston via its sealing ribs 26, 28 and 30 to seal the medicine reservoir. This lesser compressibility not only may improve dose accuracy, but also may shorten the dynamic response, and therefore the injection hold time, during which the piston's elastic properties cause the piston to return toward its original shape to account for any piston compression during injection. In one form, core 40 is a solid aluminum casting, having a hardness which is greater than the hardness of the elastomeric formulation of body 21 so as to provide a stiffening effect to the piston. Although aluminum is described as being used, one or more other relatively stiff materials, such as various plastics or other polymeric materials, may be used to form the rigid core in alternate embodiments. The hardness or resistance to deformation of these other possible core materials is greater than the hardness of the piston main body material, regardless of, for example, numerical values that are dependent on the type of hardness test, such as Brinell, Rockwell, Shore or other method, by which the hardness is measured. Furthermore, the shape of the rigid core need not be cylindrical to provide advantageous piston properties. For example, cores which have an overall shape more hour-glass in design, or cores having protruding ribs or molding features, may be used within the scope of the invention.

Core 40 can be of various axial lengths as well as various diameters, while still performing its stiffening function. For a standard piston body having an axial dimension of about 0.32 inch, and a diameter of the sealing ribs 26, 28 and 30 of about 0.39 inch, the axial length of core 40 extending between distal face 42 and proximal face 44 is within the range of about 0.08 inch-0.24 inch, more preferably within the range of about 0.16 inch-0.24 inch, and most preferably about 0.24 inch. The diameter of the core may be of various size, such as in the range of about 0.08 inch-0.31 inch.

Plug 50 covers the proximal face of core 40 and is secured to the piston body 21, such as via adhesives. Plug 50 is cylindrical and sized to completely fill the piston hollow 35 not filled by core 40, and seals core 40 within body 21. The proximal face of plug 50 is coplanar with proximal end face 24. Plug 50 is made of the same elastomeric formulation as is piston body 21. Consequently, core 40 is completely encapsulated within the elastomeric formulation that is chemically and biologically compatible with the medication contained within the pharmaceutical cartridge. In alternate embodiments, core 40 may be encapsulated within a one-piece piston body molded completely thereover, in that the plug/piston body combination shown separately formed in the embodiment of FIGS. 1-5 is integrally molded and formed as a single encapsulating piece.

The remainder of cartridge 10 may be of standard form, including a tubular barrel 55, made of glass or other suitable material, which has an inner surface 52. The inner surface 52 along the larger diameter section of the barrel is sealingly engaged by sealing ribs 26, 28 and 30 of piston 20. The distal end of tubular barrel 55 includes an inwardly sloping shoulder portion 56, a reduced diameter neck 58, and a rim 60. Rim 60 provides a circumferential flange having a larger outer radius than that of neck 58. The distal, outlet end of barrel 55 is sealed by septum 62 held by cap 64 that is secured to rim 60. The medicine-filled reservoir 54 is of variable volume due to the movability of piston 20, and is defined by septum 62, the interior surface 52, and the distal end face 22 of piston 20. In alternate embodiments, the larger diameter section of the tubular barrel can have other than the cylindrically shaped interior surface shown, provided the exterior of the piston is appropriate modified so as to provide the fluid tight seal therewith.

With reference again to FIG. 2, cartridge 10 including piston 20 is shown loaded in an abstractly shown injection pen. The injection pen is equipped with a pen-needle assembly of known design, generally designated 70, a drive member, generally designated 80, having an enlarged foot 81 that directly contacts piston 20, a manually accessible input member, such as the shown plunger, generally designated 90, and an injecting mechanism, generally designated 100. The needle of assembly 70 punctures cartridge septum 62 during mounting of the needle assembly to the medication delivery device as is conventional to provide an outlet for the pharmaceutical within the reservoir 54. As is conventional, injecting mechanism 100 is operatively connected with the drive member 80 and plunger 90 to produce appropriate motion of the drive member that shifts piston 20 distally within barrel 55 during plunger operation. A variety of different known injecting mechanisms are suitable to convert an input, such as a plunging force, into an advancement of a drive member to force medication from the pharmaceutical cartridge.

Referring now to FIG. 6, there is shown an axial cross-sectional view of an alternate cartridge piston 20′ of the present invention. In this embodiment, rigid core 100 has a larger axial length than core 40, so as to have its proximal face 101 coplanar with the annular proximal end face 24′ of the piston body 21′. Such a construction may facilitate molding, but may require either proper orientation of the piston during its assembly to the cartridge barrel during manufacture, or a suitable material selection for core 100 along with sufficient testing to satisfy any requirements related to the core material possibly being in contact with the medicine should the piston be inadvertently inverted during cartridge/piston assembly.

Piston 20 and 20′ are well suited to be axially advanced, without rotation, via an abutting contact with a foot 81 that merely translates, without rotation, during drive member 80 advancement. In alternate embodiments, piston 20 or 20′ c an be screwed into the cartridge barrel during piston advancement via, for example, a foot that contacts, and does not slip relative to, the piston, which foot and drive member screw distally as a unit during injecting operations. Such a screwably advancing piston may provide even better dose accuracy, but likely requires larger forces being generated to so move that piston during its advancement. Still further, the core and/or piston body can include a hollow for accommodating a portion of the drive member that inserts thereinto. In such a construction, the hollow may be keyed in the case of a torque-transmitting engagement between a screwing drive member and the screwable piston.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

I claim:

1. A piston for a pharmaceutical cartridge including a tubular barrel extending in an axial direction, the piston being advanceable by a separate drive member of a medication delivery device equipped with the cartridge, the piston comprising:
a body having a distal end, a proximal end and a sealing periphery, said distal end in contact with a medication disposed within the cartridge barrel, said sealing periphery in sealing contact with an interior surface of the barrel, said distal end and sealing periphery unitarily constructed from a material having a first hardness; and
a core within said body and sealed within the cartridge barrel between the distal end and the proximal end, said core constructed from at least one material having a second hardness greater than said first hardness to limit axial compressibility of said piston.

2. The piston of claim 1 wherein said core is completely encapsulated within said body.

3. The piston of claim 2 wherein said body comprises a plug that forms a portion of said proximal end and which seals an interior hollow of said body in which said core is disposed, said plug formed of the same material as said distal end of said body.

4. The piston of claim 2 wherein said core is axially centered within said body to be equidistant from said body proximal and distal ends.

5. The piston of claim 2 wherein said core is cylindrical.

6. The piston of claim 2 wherein said core axially extends between about 25% to 75% of an axial length of said body.

7. The piston of claim 6 wherein said core axially extends between about 50% to 75% of the axial length of said body.

8. The piston of claim 6 wherein said core axially extends about 75% of the axial length of said body.
